(19) United States
(12) Patent Application Publication
(10) Pub. No.: US 2011/0282221 A1
(43) Pub. Date: Nov. 17, 2011

(54) MANAGEMENT DEVICE, MANAGEMENT SYSTEM, AND MANAGEMENT METHOD

(75) Inventors: Yukiya Sawanoi, Nara-shi (JP); Yoko Shimose, Osaka (JP)

(73) Assignee: OMRON HEALTHCARE CO., LTD., Kyoto (JP)

(21) Appl. No.: 13/196,081

(22) Filed: Aug. 2, 2011

Related U.S. Application Data
(63) Continuation of application No. PCT/JP2010/050626, filed on Jan. 20, 2010.

(30) Foreign Application Priority Data
Feb. 5, 2009 (JP) 2009-025094

(51) Int. Cl. A61B 5/0225

(52) U.S. Cl. 600/493

(57) ABSTRACT

A calibration device is connected to a sphygmomanometer with an air tube and a communication cable. When connection is detected, the calibration device closes a valve of the sphygmomanometer and its valve and applies pressure to the air tube to measure the pressure, and determines the air leakage of the sphygmomanometer based on the pressure change with an air leakage determination unit. The calibration device causes a pressure sensor of the sphygmomanometer to measure the inner pressure, receives the measurement result with the communication I/F, and determines the result of the equipment difference test of the sphygmomanometer based on a difference between the applied pressure and the inner pressure with an equipment difference determination unit. If determined that the equipment difference test is failure, a control signal is output to the sphygmomanometer to calibrate the output value of the pressure sensor in the calibration unit.

### TECHNICAL FIELD

The present invention relates to management devices, management systems, and management methods, and in particular, to a management device, a management system, and a management method for carrying out management of an electronic sphygmomanometer.

### BACKGROUND ART

A blood pressure is one index for analyzing a circulatory disease, where performing risk analysis based on the blood pressure is effective in preventing cardiovascular diseases such as stroke, heart failure, and cardiac infarction.

Conventionally, diagnosis is made from the blood pressure (occasional blood pressure) measured in medical institutions such as at the time of hospital visit, health check or the like. However, it is found from researches of recent years that the blood pressure (home blood pressure) measured at home is more useful in diagnosis of circulatory diseases than the occasional blood pressure. Accordingly, an electronic sphygmomanometer used at home is being widely used.

In using the sphygmomanometer at home, a user may not know whether or not the measurement accuracy of the sphygmomanometer is accurate. The sensor for detecting pressure influences the measurement accuracy of the sphygmomanometer the most. Each sensor has different characteristics, and hence, a calibration complying with the characteristics of an individual sensor is required at the time of factory shipment, or the like. A technique for easily achieving the calibration of the sensor is disclosed in Japanese Unexamined Patent Publication No. 7-51233 (Japanese Patent No. 3178175) (patent document 1), which is a patented invention by the applicant of the present application. In this patented invention, a plurality of patterns of a relationship of the difference between an application pressure value and a detection pressure value is stored in advance, and a pattern close to the relationship of the actual difference is selected and set in the nonvolatile memory of the sphygmomanometer to easily carry out the calibration of the sensor.
Patent Document 1: Japanese Unexamined Patent Publication No. 7-51233

### SUMMARY OF INVENTION

However, the blood pressure constantly fluctuates by various environmental factors such as stress, time, meal, and exercise. Thus, the measurement result may differ between the occasional blood pressure and the home blood pressure, or the blood pressure value may differ for every measurement even if the home blood pressure is repeatedly measured. At home, determination cannot be made whether the difference in the blood pressure value is due to an environmental factor or due to the measurement accuracy of the electronic sphygmomanometer.

When using at home, the user is not able to know whether or not the measurement accuracy of the electronic sphygmomanometer is accurate, and thus may feel a sense of insecurity to the measurement accuracy of the electronic sphygmomanometer if the blood pressure value differs. Therefore, some users send the electronic sphygmomanometer to the manufacturing company to check whether or not the electronic sphygmomanometer is defective. The blood pressure cannot be measured while the electronic sphygmomanometer is being sent to the manufacturing company. Some users may feel a sense of uncertainty to the measurement accuracy of the electronic sphygmomanometer and may not carry out the measurement. If the home blood pressure is not obtained, information useful for the diagnosis of the circulatory disease reduces.

Therefore, one or more embodiments of the present invention provides a management device, a management system, and a management method enabling a function test of the electronic sphygmomanometer to be easily carried out and a calibration to be carried out without special knowledge.

According to one or more embodiments of the present invention, a management device is a management device for performing management of an electronic sphygmomanometer for detecting an inner pressure change of an air bladder with a sensor and calculating a blood pressure value based on an output value of the sensor; the management device including a connecting unit for connecting to the electronic sphygmomanometer; a test unit for testing an equipment performance of the electronic sphygmomanometer while being connected to the electronic sphygmomanometer with the connecting unit; a calibration unit for calibrating the equipment performance of the electronic sphygmomanometer according to the test result of the test unit; and a first output unit for outputting the test result of the test unit or presence or absence of calibration in the calibration unit.

According to one or more embodiments of the present invention, a management system includes an electronic sphygmomanometer for detecting an inner pressure change of an air bladder with a sensor and calculating a blood pressure value based on an output value of the sensor; and a management device, connected to the electronic sphygmomanometer, for managing the electronic sphygmomanometer; wherein the management device includes, a test unit for carrying out an operation for testing an equipment performance of the electronic sphygmomanometer while being connected to the electronic sphygmomanometer, a calibration unit for calibrating the equipment performance of the electronic sphygmomanometer according to the test result of the test unit, and an output unit for outputting the test result of the test unit or presence or absence of calibration in the calibration unit; and the electronic sphygmomanometer includes, a drive unit for operating the electronic sphygmomanometer according to a control signal output in the test unit, a measurement unit for transmitting a signal corresponding to the output value of the sensor to the management device with the operation, and a changing unit for changing a relationship between the sensor signal from the sensor and the output value of the sensor according to a control signal output in the calibration unit.

According to one or more embodiments of the present invention, a management method is a management method of an electronic sphygmomanometer in a management system including, an electronic sphygmomanometer for detecting an inner pressure change of an air bladder with a sensor and calculating a blood pressure value based on an output value of the sensor; and a management device, connected to the electronic sphygmomanometer, for managing the electronic sphygmomanometer; the method including the steps of having the management device detect connecting with the electronic sphygmomanometer, and carry out an operation for testing an equipment performance of the electronic sphygmomanometer while being connected to the electronic sphygmomanometer; the electronic sphygmomanometer operating according to a control signal output from the management device in a step of carrying out the operation for testing the equipment performance; the electronic sphygmomanometer transmitting a signal corresponding to an output value of the sensor to the management device with the operation; the management device determining the equipment performance of the electronic sphygmomanometer based on the signal transmitted from the electronic sphygmomanometer and/or value detected in the connected state; the management device calibrating the equipment performance of the electronic sphygmomanometer according to the determination, the electronic sphygmomanometer changing a relationship between the sensor signal from the sensor and the output value of the sensor according to a control signal output from the management device in the step of calibrating the equipment performance; and the management device outputting the test result of air leakage, test result of the accuracy of the output value of the sensor, or presence or absence of calibration of the output value of the sensor.

According to one or more embodiments the present invention, the function test of the electronic sphygmomanometer can be easily carried out without expert knowledge on the equipment configuration of the electronic sphygmomanometer, and calibration can be carried out. Therefore, the measurement result of the sphygmomanometer becomes reliable, the blood pressure measurement can be continuously carried out as a result even at home, and home blood pressure that is useful information in diagnosing the circulatory disease can be obtained.

### BRIEF DESCRIPTION OF THE DRAWINGS

FIG. 1 is a view showing a specific example of a configuration of a calibration system according to one or more embodiments of the present invention and a configuration of each device contained in the calibration system.

FIG. 2 is a view describing a calibration of a pressure sensor arranged in a sphygmomanometer.

FIG. 3 is a view describing a calibration of a pressure sensor arranged in a sphygmomanometer.

FIG. 4 is a flowchart showing a specific example of a flow of operations carried out in the calibration system.

FIG. 5 is a flowchart showing the flow of operation in the air leakage test during the operation of FIG. 4.

FIG. 6 is a flowchart showing the flow of operation in the equipment difference test during the operation of FIG. 4.

FIG. 7 is a view showing a specific example of a display screen of the test result.

FIG. 8 is a view showing a specific example of a display screen of the test result.

FIG. 9 is a view showing a specific example of a display screen of the test result.

FIG. 10 is a view showing a specific example of a display screen of the test result.

FIG. 11 is a view showing a specific example of a screen displaying the record of the calibration.

FIG. 12 is a view showing a specific example of a screen urging the next test and calibration.

FIG. 13 is a view showing another specific example of the configuration of the calibration system according to one or more embodiments of the present invention and the configuration of each device arranged in the calibration system.

### DETAILED DESCRIPTION OF INVENTION

Embodiments of the present invention will be hereinafter described with reference to the drawings. In the following description, the same reference numerals are denoted for the same components and configuring elements. The names and functions thereof are also the same.

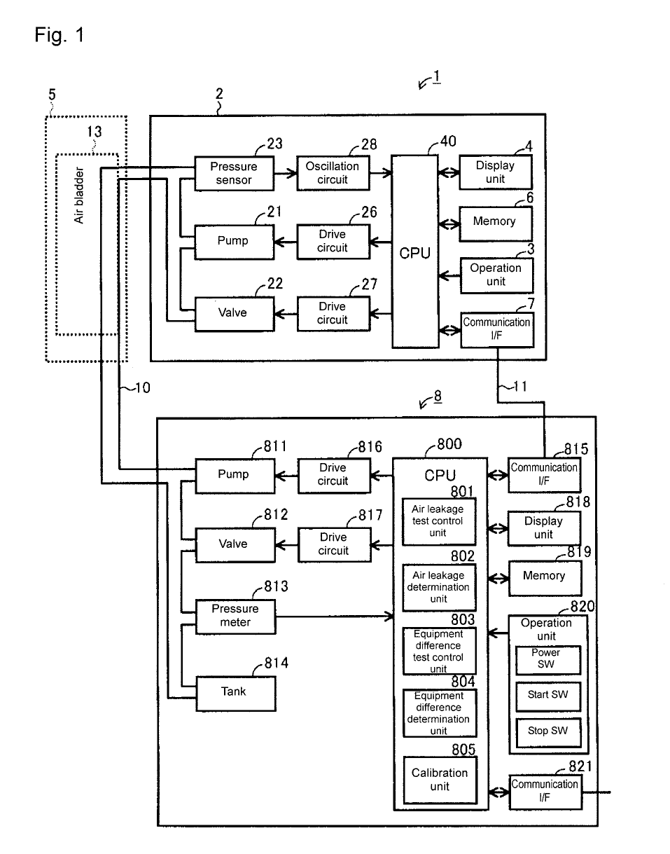

FIG. 1 is a view showing a specific example of a configuration of a calibration system according to one or more embodiments of the present invention as a system for managing an electronic sphygmomanometer and a configuration of each device contained in the calibration system. With reference to FIG. 1, the calibration system includes an electronic sphygmomanometer (hereinafter referred to as sphygmomanometer 1), and a calibration device 8 or a management device for testing the sphygmomanometer 1 and calibrating as necessary, as described later. The sphygmomanometer 1 and the calibration device 8 are electrically connected with a communication cable 11 to perform a two-way communication. The communication between the sphygmomanometer 1 and the calibration device 8 includes communication complying with the standard such as RS-232 (Recommended Standard 232), but may be other communications. The communication is not limited to wired communication and may be wireless communication such as infrared communication.

The sphygmomanometer 1 includes a main body portion 2 and is connected to an air bladder 13 included in a cuff 5 with an air tube 10 at the time of blood pressure measurement. In addition to the connection to the calibration device 8 with the communication cable 11 at the time of test described later, connection is made to the calibration device 8 with the air tube 10 in place of the air bladder 13. The cuff 5 is wrapped around the upper arm that is the measurement site. An operation unit 3 including a switch for instructing the start of measurement and a display unit 4 for displaying measurement results and the like are arranged on the front surface of the main body portion 2.

The main body portion 2 includes a pressure sensor 23 for measuring a change in inner pressure of the air bladder 13, a pump 21, and a valve 22 connected to the air bladder 13 with the air tube 10 in between. The pressure sensor 23, the pump 21, and the valve 22 are respectively connected to an oscillation circuit 28, a drive circuit 26, and a drive circuit 27, and in turn, the oscillation circuit 28, the drive circuit 26, and the drive circuit 27 are respectively connected to a CPU (Central Processing Unit) 40 for controlling the entire sphygmomanometer 1.

The CPU 40 is also connected to the display unit 4, the operation unit 3, a memory 6, and a communication interface (hereinafter abbreviated as I/F) 7. The memory 6 stores control programs executed by the CPU 40, measurement results, test results to be described later, and the like. The memory 6 also becomes a work region when the CPU 40 executes the program. The communication I/F 7 is an interface connecting to the calibrating device 8 with the communication cable 11 for communication.

The control program stores a measurement program for carrying out a normal blood pressure measurement operation, and a calibration program, to be described later, for realizing a mode (hereinafter referred to as calibration mode) of being subjected to test and calibration based on the command of the calibration device 8. When the CPU 40 reads out and executes the measurement program, the sphygmomanometer 1 enters a mode (hereinafter referred to as normal mode) of performing the measurement operation, and the blood pressure measurement operation is carried out according to the operation signal or the like from the operation unit 3. When the CPU 40 reads out and executes the calibration program, the sphygmomanometer 1 enters a calibration mode, and the test and calibration operation is carried out by operating each unit according to the command received from the calibration device 8 at the communication I/F 7.

The CPU 40 executes a predetermined program stored in the memory 6 based on the operation signal input from the operation unit 3, and outputs a control signal to the drive circuit 26 and the drive circuit 27. The drive circuit 26 and the drive circuit 27 drive the pump 21 and the valve 22 according to the control signal. The pump 21 has the drive controlled by the drive circuit 26 according to the control signal from the CPU 40 to inject air into the air bladder 13. The valve 22 has the opening and closing thereof controlled by the drive circuit 27 according to the control signal from the CPU 40 to exhaust air in the air bladder 13.

The pressure sensor 23 is a capacitance sensor, where a capacitance value changes by the change in inner pressure of the air bladder 13. The oscillation circuit 28 inputs a signal of an oscillating frequency corresponding to the capacitance value of the pressure sensor 23 to the CPU 40.

The CPU 40 stores a coefficient in advance, and determines the inner pressure of the air bladder 13 that is the sensor output value from the signal from the pressure sensor 23 and the relevant coefficient. The CPU 40 executes a predetermined process based on the change in inner pressure of the air bladder 13 obtained from the pressure sensor 23, and outputs the control signal to the drive circuit 26 and the drive circuit 27 according to the result thereof. The CPU 40 performs a process of calculating the blood pressure value based on the change in inner pressure of the air bladder 13 obtained from the pressure sensor 23, and displaying the measurement result on the display unit 4, and outputs the data and the control signal for display to the display unit 4.

The air tube 10 is attachable to the calibration device 8, where the calibration device 8 is connected to the sphygmomanometer 1 with the air tube 10 by connecting the air tube 10 to the calibration device 8. The calibration device 8 includes a pump 811, a valve 812, a pressure meter 813, and a tank 814. The tank 814 may be a substitute of a cuff when performing test or calibration of the sphygmomanometer 1. If the air tube 10 is connected to the calibration device 8, the pump 811, the valve 812, the pressure meter 813, and the tank 814 of the calibration device 8 are connected to the pressure sensor 23, the pump 21, and the valve 22 of the sphygmomanometer 1 with the air tube 10 in between thus configuring one closed space.

The pump 811 and the valve 812 are respectively connected to a drive circuit 816 and a drive circuit 817, and the drive circuit 816 and the drive circuit 817 are in turn connected to a CPU 800 for controlling the entire calibration device 8. The pressure meter 813 is also connected to the CPU 800.

The CPU 800 is connected to a display unit 818, an operation unit 820, a memory 819, and communication I/Fs 815, 821. The memory 819 stores a control program or the like to be executed by the CPU 800. Furthermore, the memory 819 also becomes a work region when the CPU 800 executes the program. The communication I/F 815 is an interface connecting to the sphygmomanometer 1 with the communication cable 11 for communication. The communication I/F 821 is an interface for communicating with other devices using the communication function if the calibration device 8 has the communication function such as communication through Internet.

The operation unit 820 includes a power switch for instructing ON/OFF of the power supply, a start switch for instructing start of the test operation, and a stop switch for instructing stop of the test operation, as will be described later.

The CPU 800 executes a predetermined program stored in the memory 819 based on an operation signal input when the switch arranged in the operation unit 820 is pushed, and outputs a control signal to the drive circuit 816 and the drive circuit 817. The drive circuit 816 and the drive circuit 817 drive the pump 811 and the valve 812 according to the control signal. The pump 811 has the drive controlled by the drive circuit 816 according to the control signal from the CPU 800 to inject air into the closed space if the air tube 10 is connected to the calibration device 8. The valve 812 has the opening and closing thereof controlled by the drive circuit 817 according to the control signal from the CPU 40 to exhaust air in the closed space. The pressure meter 813 includes a pressure sensor similar to the sphygmomanometer 1, and measures the inner pressure of the closed space if the air tube 10 is connected to the calibration device 8 and inputs the measurement result to the CPU 800.

The CPU 800 includes a leakage air test control unit 801, an air leakage determination unit 802, an equipment difference test control unit 803, an equipment difference determination unit 804, and a calibration unit 805. It is shown in FIG. 1 that these are functions mainly formed in the CPU 800 when the CPU 800 reads out and executes the control program stored in the memory 819 according to the operation signal from the operation unit 820, but at least some of such functions may be formed to include one of the hardware configurations shown in FIG. 1.

The air leakage test control unit 801 controls the operation for air leakage test to be described later. The air leakage determination unit 802 determines the presence or absence of air leakage based on the air leakage amount obtained as a result of the air leakage test. The equipment difference test control unit 803 controls the operation for the equipment difference test according to the determination result of the air leakage determination unit 802. The equipment difference is defined as an error of a measuring equipment in the measurement law, or the like, and specifically corresponds to a value obtained by subtracting the true value from the measurement value. The equipment difference determination unit 804 determines success/failure of the equipment difference test based on the equipment difference obtained as a result of the equipment difference test. The calibration unit 805 performs calibration to correct the output value of the pressure sensor 23 of the sphygmomanometer 1 according to the result of the equipment difference test. As hereinafter described, the calibration refers to a process of correcting the function of the sensor output with respect to the application pressure of the pressure sensor 23, and specifically refers to a process of changing the coefficient to use to obtain the sensor output value by a signal from the pressure sensor 23 in the CPU 40 of the sphygmomanometer 1. The calibration unit 805 generates and outputs the control signal for changing the coefficient with respect to the sphygmomanometer 1.

The calibration device 8 is connected to the sphygmomanometer 1 with the air tube 10 and the communication cable 11, and tests the equipment performance of the sphygmomanometer 1. For the test of the equipment performance, an example of performing the test of leakage of air (air leakage) in the sphygmomanometer 1 and the test of equipment difference representing the accuracy of the pressure sensor will be described. The calibration device 8 calibrates the pressure sensor 23 according to the result of the equipment difference test. Other tests of the equipment performance include inputting a pseudo pulse wave signal to the sphygmomanometer 1 and testing whether or not the blood pressure calculation operation is correctly performed.

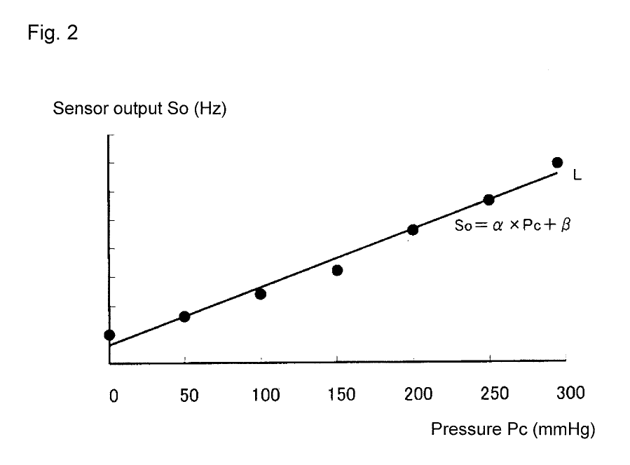

Generally, the characteristics of the pressure sensor are not constant, and the sensor output (frequency) may not necessarily change linearly as shown with dots in FIG. 2 even if the application pressure is changed linearly.

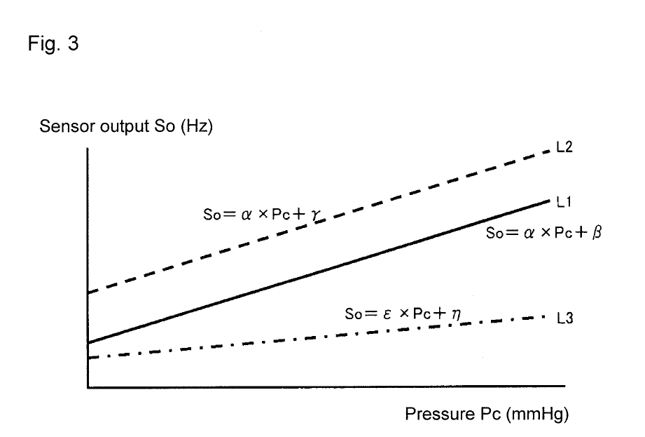

The sensor characteristics of the pressure sensor change as the pressure sensor changes over the years. In other words, a line L1 in FIG. 3 represents the sensor characteristics set at the time of shipment, whereas lines L2, L3 represent the sensor characteristics after the change of the pressure sensor over the years. The change in the sensor characteristics shown with the line L2 is offset changed with respect to the original sensor characteristics set at the time of shipment, and the sensor output is a constant change regardless of the application pressure. The change in the sensor characteristics shown with the line L2 may be corrected by offsetting the sensor output at the time of opening to atmospheric pressure so as to take a defined output value corresponding to the original sensor characteristics stored in advance in the initialization process executed at the time of power ON. In the change in the sensor characteristics shown with the line L3, the proportion of change of the sensor output differs according to the application pressure in addition to the offset change. In other words, the change in the sensor characteristics shown with the line L3 includes a change of tilt corresponding to the change of the sensor output with respect to the change of the application pressure in addition to the offset change.

The calibration device 8 determines coefficients α and β of an approximate line obtained from the actual sensor outputs in which the relationship of the sensor output with respect to the application pressure is represented with a line L in FIG. 2 with respect to the variation of the sensor output with respect to the application pressure of the pressure sensor 23 of the sphygmomanometer 1 shown in FIG. 2. The coefficients ε and η of an approximate line are determined similar to the above with respect to the sensor characteristics of after the change of the pressure sensor over the years represented with an approximate line L3 of FIG. 3.

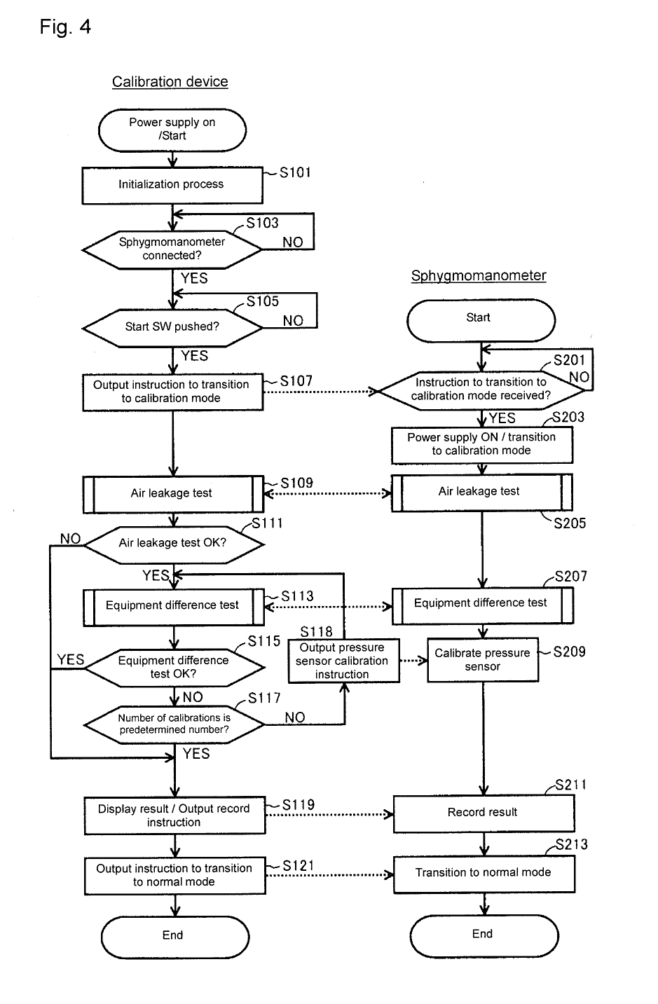

The flowchart on the left side of FIG. 4 is the operation of the calibration device 8, and the flowchart on the right side is the operation of the sphygmomanometer 1. Such operations are realized when the CPU of each device reads out and executes the program stored in the memory, and controls each unit shown in FIG. 1. The operation shown in the flowchart on the left side of FIG. 4 starts when the power switch in the operation unit 820 of the calibration device 8 is pushed and the power supply is turned ON.

With reference to FIG. 4, when the power supply of the calibration device 8 is turned ON, the work region of the memory 819 is initialized and the initialization process of performing 0 mmHg adjustment, or the like of the pressure meter 813 is performed in step S101, and thereafter, whether or not the sphygmomanometer 1 is connected with the air tube 10 is monitored (step S103). This may be realized with a mechanism of arranging a switch (not shown) at the connecting portion with the air tube 10 of the calibration device 8 so that the switch is pushed when the air tube 10 is attached. A storage device such as an IC chip and a reading device may be arranged at the contact portion of the air tube 10 and the main body of the calibration device 8, and the CPU 800 may determine that connection is established when detecting the communication in between.

The sphygmomanometer 1 is connected when the air tube 10 is connected to the calibration device 8 (YES in step S103), where if the operation signal indicating the pushing of the start switch instructing the start of the test operation is input from the operation unit 820 in such state (YES in step S105), the CPU 800 transmits a command to shift the sphygmomanometer 1 to the calibration mode from the communication I/F 815 to the sphygmomanometer 1 in step S107.

In the sphygmomanometer 1, when receiving the command transmitted in step S107 from the calibration device 8 with the communication I/F 7 (YES in step S201), the CPU 40 turns ON the power supply, reads out the calibration program from the memory 6 according to the command and executes the same, and shifts the operation mode to the calibration mode in step S203. In step S203, the CPU 40 may automatically turn ON the power supply, or a screen urging the operation such as “please turn ON power” stored in advance may be displayed on the display unit 818 of the calibration device 8 so that the power switch of the operation unit 3 of the sphygmomanometer 1 is operated. The shift to the calibration mode may be carried out when the CPU 40 automatically reads out the calibration program according to the control signal, or may be carried out when the CPU 40 detects one of application of a predetermined pressure pattern to the air tube 10 from the calibration device 8 in place of the control signal, application of a power supply voltage to supply to the sphygmomanometer 1 in a predetermined voltage pattern, reception of operation of the switch if the sphygmomanometer 1 includes such dedicated switch, or operation of the switch of the operation unit 3 in a predetermined pattern with the control signal.

In step S109, the air leakage test control unit 801 performs the control in the calibration device 8 to perform the operation for the air leakage test. The air leakage test is realized when a predetermined operation is carried out in step S205 of the sphygmomanometer 1 with the operation of the calibration device 8.

In step S111, the air leakage determination unit 802 determines whether or not the result of the air leakage test in steps S109 and S205 is appropriate, that is, whether or not there is air leakage in the sphygmomanometer 1 main body. If the air leakage test is OK, that is, if determined that there is no air leakage in the sphygmomanometer 1 main body (YES in step S111), the equipment difference test control unit 803 performs the control to carry out the operation for the equipment difference test in the calibration device 8 in step S113. The equipment difference test is realized when a predetermined operation is carried out in step S207 of the sphygmomanometer 1 with the operation of the calibration device 8. The result of the operation in the sphygmomanometer 1 in step S207 is transmitted to the calibration device 8.

In step S115, the equipment difference determination unit 804 determines whether or not the result of the equipment difference test in steps S113, S207 is appropriate, that is, whether or not the equipment difference of the sphygmomanometer 1 is within a tolerable range. If the equipment difference test is not OK, that is, if the output value obtained in the test exceeds a tolerable range from the “true value” with the application output value as the “true value” (NO in step S115), and the number of executions of the operation for calibrating the pressure sensor 23 to be described later does not meet the defined predetermined number (NO in step S117), the calibration unit 805 transmits a control signal for causing the sphygmomanometer 1 to execute the calibration operation for calibrating the pressure sensor 23 to the sphygmomanometer 1. In step S118, the calibration unit 805 may transmit the predefined control signal to update the coefficient the CPU 40 uses to obtain the sensor output value from the signal from the pressure sensor 23 by a predetermined amount stored in advance, or may calculate the update amount of the coefficient from the equipment difference obtained in the equipment difference test of step S113 and transmit a control signal to update by such amount.

In the sphygmomanometer 1, when receiving the control signal transmitted in step S118 from the calibration device 8 with the communication I/F 7, the CPU 40 executes the calibration operation in step S209. In other words, the CPU 40 updates the coefficient to use to obtain the sensor output value from the signal from the pressure sensor 23 according to a control signal to correct and calibrate the sensor output value of the pressure sensor 23.

After the calibration operation of steps S118, S209 is carried out, the equipment difference test of steps S113, S207 is again executed to check the function of the pressure sensor 23 after the calibration, and the calibration operation is further carried out according to the test result. The number of calibration operations of steps S118, S209 is defined in advance, where the CPU 800 determines as failure of the pressure sensor 23 of the sphygmomanometer 1 if the equipment difference test is not OK even after the calibration of step S118 is carried out a predetermined number of times (NO in step S115 and YES in S117).

After the operation for a series of tests is finished, the CPU 800 performs a process of displaying a screen displaying the above test result on the display unit 818 to display on the display unit 818 in step S119. The control signal for storing the test results and the record of calibration in the memory 6 of the sphygmomanometer 1 is also generated and transmitted to the sphygmomanometer 1 with the information to be stored. In the sphygmomanometer 1, in step S211, the CPU 40 performs a process of storing the transmitted test results and the record of calibration in a predetermined region of the memory 6 according to the control signal transmitted from the calibration device 8 in step S119. In this case, the test results and the record of calibration may be displayed in the display unit 4.

The test results and the record of calibration may be stored on the calibration device 8 side as well. In other words, in step S119, the CPU 800 may store the test results and the record of calibration in a predetermined region of the memory 819 along with the information (e.g., serial number, user name registered in advance, etc.) for specifying the sphygmomanometer 1. The information for specifying the sphygmomanometer 1 may be acquired when the CPU 800 automatically makes a request to the sphygmomanometer 1 upon detecting the connection of the communication cable 11 of the sphygmomanometer 1 in step S103, may be automatically read from a predetermined region of the memory 6, or a screen urging the input may be displayed on the display unit 818 at the relevant timing and acquired when receiving input from a key (not shown) and the like of the operation unit 820.

According to one or more embodiments of the present invention, when determined that there is air leakage in the test, or when determined that the equipment difference is outside the tolerable range and the calibration of the pressure sensor 23 is carried out, the CPU 40 adds at least information indicating that there is possibility the accuracy may not be satisfactory to the measurement value stored in the memory 6 from the previous test or the date and time of the calibration to the operation of this time. Thus, when reading out such measurement value and using it in diagnosis, such value may not be used thus enhancing the reliability of the measurement value in the sphygmomanometer 1.

Thereafter, in step S121, the CPU 800 transmits a command for having the sphygmomanometer 1 in the normal mode from the communication I/F 815 to the sphygmomanometer 1, and terminates the series of operations. In the sphygmomanometer 1, when receiving the command transmitted in step S121 from the calibration device 8 with the communication I/F 7, the CPU 40 executes the measurement program from the memory 6 according to the command in step S213 to shift the operation mode to the normal mode, and terminates the operation in the series of calibration mode.

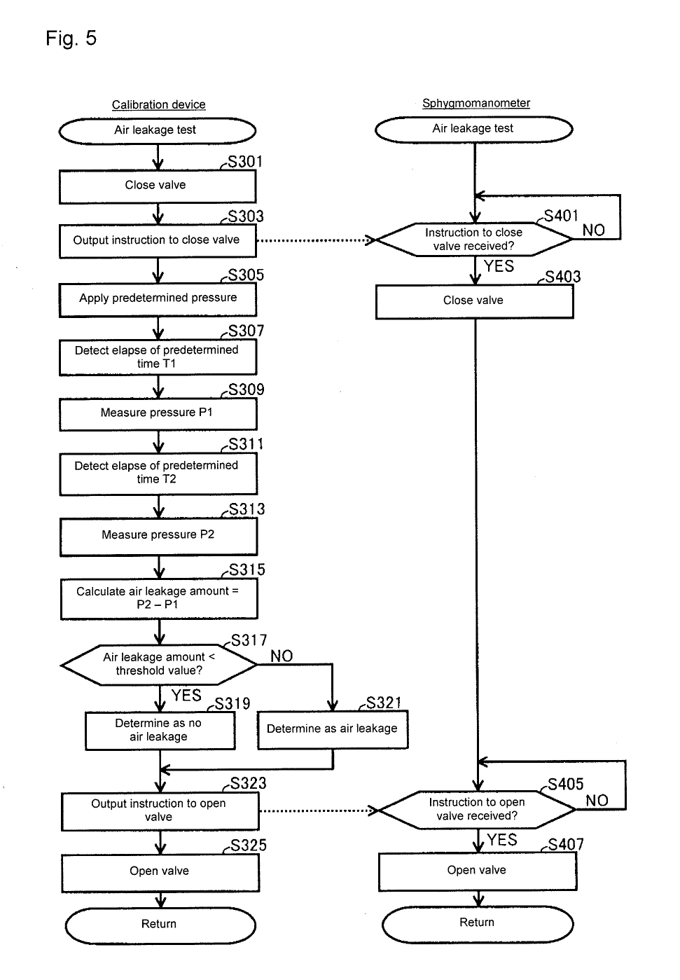

FIG. 5 is a flowchart showing the flow of operation in the air leakage test in steps S109, S205, and similarly, the flowchart on the left side shows the operation in the calibration device 8 and the flowchart on the right side shows the operation in the sphygmomanometer 1. The air leakage test here adopts a test method defined in the accuracy standard (SP10) of AAMI (Association for the Advancement of Medical Instrumentation) in the United States or a test method defined in JIS T4203-1990.

With reference to FIG. 5, when the operation for the air leakage test starts, the air leakage test control unit 801 of the CPU 800 outputs a control signal to the drive circuit 817 to close the valve 812 in step S301. In step S303, the air leakage test control unit 801 generates a control signal for blocking the valve, and outputs the same to the sphygmomanometer 1 from the communication I/F 815.

In the sphygmomanometer 1 that shifted to the calibration mode in step S203, when receiving the control signal transmitted in step S303 from the calibration device 8 with the communication I/F 7 (YES in step S401), the CPU 40 outputs a control signal to the drive circuit 27 according to the control signal to close the valve 22 in step S403.

In step S305, the air leakage test control unit 801 outputs a control signal to the drive circuit 816 to apply a predetermined pressure to the pressure sensor 23 of the sphygmomanometer 1, and drives the pump 811 to inject air of an amount corresponding to the predetermined pressure to the tank 814 and the air tube 10. When the air leakage test control unit 801 detects elapse of a predetermined time T1 (step S307) after applying a predetermined pressure by injecting a predetermined amount of air into the air tube 10, the pressure P1 in the tank 814 and the air tube 10 is measured with the pressure meter 813 in step S309. When the air leakage test control unit 801 further detects elapse of a predetermined time T2 thereafter (step S311), the pressure P2 in the air tube 10 is measured with the tank 814 and the pressure meter 813 in step S313.

According to step S301 and step S403, in the sphygmomanometer 1 and the calibration device 8 connected with the air tube 10, a space closed with the air tube 10 connected with the pump 21, the valve 22, and the pressure sensor 23 of the sphygmomanometer 1, and the pump 811, the valve 812, the pressure meter 813, and the tank 814 of the calibration device 8 is thereby configured. Therefore, the pressure P1 measured in step S309 and the pressure P2 measured in step S313 are also considered as pressure inside the sphygmomanometer 1.

In step S315, the air leakage test control unit 801 calculates the air leakage amount by subtracting the pressure P1 obtained in step S309 from the pressure P2 obtained in step S313. In step S315, the difference of the inner pressure (P1) of after elapse of time T1 and the inner pressure (P2) of after elapse of time T2 thereafter is calculated as the air leakage amount assuming the change in pressure from after elapse of time T1 to elapse of time T2 is due to air leakage.

In step S317, the air leakage determination unit 802 compares the difference in pressure calculated as the air leakage amount in step S315 with the threshold value complying with the above standard stored in advance, and determines that there is not air leakage in the main body of the sphygmomanometer 1 (step S319) if the difference is smaller than the threshold value (YES in step S317). If not (NO in step S317), determination is made that there is air leakage in the main body of the sphygmomanometer 1 (step S321).

After the series of operations above is completed, the air leakage test control unit 801 generates a control signal for opening the valve and outputs the same to the sphygmomanometer 1 from the communication I/F 815 in step S323. When receiving the control signal transmitted in step S323 from the calibration device 8 with the communication I/F 7 (YES in step S405), the CPU 40 outputs a control signal to the drive circuit 27 according to such control signal to open the valve 22 in step S407.

In step S325, the air leakage test control unit 801 outputs a control signal to the drive circuit 817 to open the valve 812, and terminates the operation for the series of air leakage test.

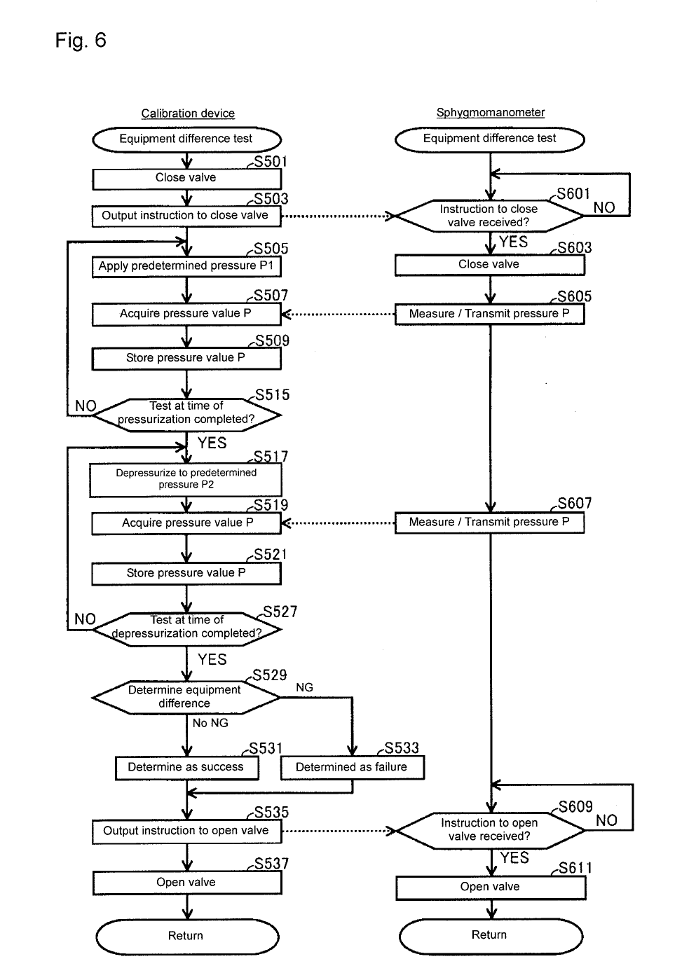

FIG. 6 is a flowchart showing the flow of operation in the equipment difference test in steps S113, S207, and similarly, the flowchart on the left side shows the operation in the calibration device 8 and the flowchart on the right side shows the operation in the sphygmomanometer 1. The equipment difference test may adopt the test method defined in JIS T1115-2005.

With reference to FIG. 6, when the operation for the equipment difference test starts, the equipment difference test control unit 803 of the CPU 800 outputs a control signal to the drive circuit 817 to close the valve 812 in step S501. In step S303, the equipment difference test control unit 803 generates a control signal for closing the valve and outputs the same to the sphygmomanometer 1 from the communication I/F 815.

In the sphygmomanometer 1 that shifted to the calibration mode in step S203, when receiving the control signal transmitted in step S503 from the calibration device 8 with the communication I/F 7 (YES in step S601), the CPU 40 outputs a control signal to the drive circuit 27 according to the control signal to close the valve 22 in step S603.

In step S505, the equipment difference test control unit 803 outputs a control signal to the drive circuit 816 to apply a predetermined pressure P1 to the pressure sensor 23 of the sphygmomanometer 1, and drives the pump 811 to inject air of an amount corresponding to the pressure P1 to the tank 814 and the air tube 10. When the predetermined pressure P1 is applied by injecting a predetermined amount of air to the tank 814 and the air tube 10, the equipment difference test control unit 803 generates a control signal for measuring the inner pressure of the tank 814 and the air tube 10 in the sphygmomanometer 1 and outputs the same to the sphygmomanometer 1 from the communication I/F 815.

In the sphygmomanometer 1, when receiving the control signal from the calibration device 8 with the communication I/F 7, the CPU 40 obtains the sensor output value using the signal from the pressure sensor 23 and the coefficient and outputs the inner pressure P measured in the sphygmomanometer 1 represented with the sensor output value to the calibration device 8 from the communication I/F 7 in step S605. In step S507, the calibration device 8 acquires the inner pressure P that is the measurement value transmitted from the sphygmomanometer 1 in step S605.

In step S509, the equipment difference test control unit 803 stores the inner pressure P that is the measurement value in the sphygmomanometer 1 received and acquired from the sphygmomanometer 1 in step S507 in a predetermined region of the memory 819 in association with the pressure value P1 applied in step S505.

If the test method defined in JIS T1115-2005 is performed for the equipment difference test, the above operations are repeated while pressurizing at a predetermined pressure interval. Specific examples of the pressure P1 to be applied include 0, 50, 100, 150, 200, 250, 295 mmHg. In other words, if the pressurization has not reached the upper limit of the test at the time of pressurization stored in advance after step S511 or step S513 (NO in step S515), the pressurized pressure P1 is further applied at the predetermined pressure interval and the operations after step S505 are repeated.

If the pressurization has reached the upper limit and the test at the time of pressurization is completed (YES in step S515), similar operation is repeated while depressurizing at a predetermined pressure interval if the test method defined in JIS T1115-2005 is performed for the equipment difference test. In other words, the equipment difference test control unit 803 outputs a control signal for measuring the inner pressure P with respect to the sphygmomanometer 1 after depressurizing the inner pressure of the air tube 10 to a predetermined pressure P2 in step S517, so that the measured inner pressure P is acquired from the sphygmomanometer 1 in step S519 and the inner pressure P or the measurement value in the sphygmomanometer 1 is stored in a predetermined region of the memory 819 in association with the pressure P2 applied in step S517 in step S523. The operation at the time of depressurization is also repeated until reaching the lower limit pressure (YES in step S527) at a predetermined pressure interval similar to the time of pressurization.

Through the above operations, the pressure P measured in the sphygmomanometer 1 for every pressure P1 at the time of pressurization and the pressure P measured in the sphygmomanometer 1 for every pressure P2 at the time of depressurization are stored in the predetermined region of the memory 819 of the calibration device 8. According to one or more embodiments of the present invention, if the test method defined in JIS T1115-2005 is performed for the equipment difference test, the operations of steps S505 to S515 and S517 to S527 are carried out two times each.

Using the values stored through the above operations, the equipment difference test control unit 803 calculates the equipment difference in step S529 and the equipment difference determination unit 804 determines whether or not such equipment difference is within a tolerable range. In other words, in step S529, the equipment difference test control unit 803 calculates the difference from the “true value” of the inner pressure P measured in the sphygmomanometer 1 as the equipment difference with the applied pressures P1, P2 as the “true value” for the time of pressurization and for the time of depressurization, respectively. The equipment difference determination unit 804 compares the calculated equipment difference with the acceptable value stored in advance and determines whether or not the equipment difference is smaller than or equal to the acceptable value. According to one or more embodiments of the present invention, if the test method defined in JIS T1115-2005 is performed for the equipment difference test, such determination is carried out using the average value of two times of the equipment difference for the time of pressurization and for the time of depressurization. The equipment difference determination unit 804 determines that the equipment difference test is success (step S531) if determined that the equipment difference is smaller than or equal to the acceptable value compared to the acceptable value of the equipment differences obtained in the series of operations, that is, all the equipment differences are within the tolerable range (no NG in step S529). If even one equipment difference is greater than the acceptable value, that is, if even one equipment difference is outside the tolerable range (NG in step S529), the equipment difference determination unit 804 determines that the equipment difference test is failure (step S533).

After the series of operations are completed, the equipment difference test control unit 803 generates a control signal for opening the valve and outputs the same to the sphygmomanometer 1 from the communication I/F 815 in step S535. When receiving the control signal transmitted in step S535 from the calibration device 8 with the communication I/F 7 (YES in step S609), the CPU 40 outputs a control signal to the drive circuit 27 according to the control signal to open the valve 22 in step S611.

In step S537, the equipment difference test control unit 803 outputs the control signal to the drive circuit 817 to open the valve 812, and terminates the operation for the series of equipment difference tests.

When the above operations are executed in the calibration system including the sphygmomanometer 1 and the calibration device 8, the user of the sphygmomanometer 1 can easily carry out the function test of the sphygmomanometer 1 by simply connecting the air tube 10 and the communication cable 11 to the calibration device 8 and operating the start switch even if the user does not have expert knowledge.

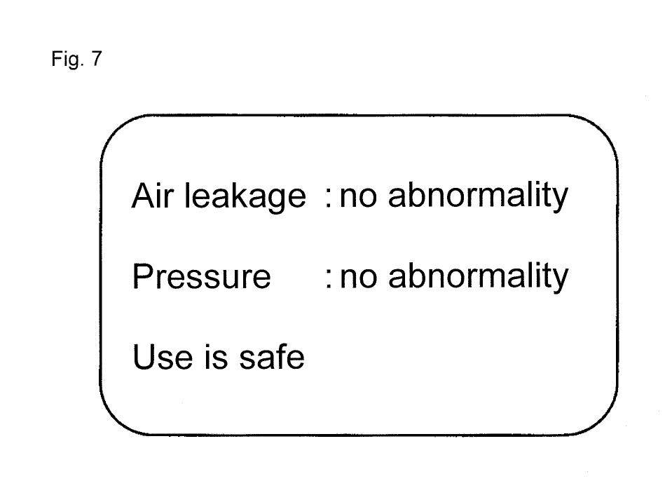

If determined that there is not air leakage in the main body of the sphygmomanometer 1 in the air leakage test of steps S109, S205 in step S111 and if determined that the equipment difference of the sphygmomanometer 1 is within the tolerable range in the equipment difference test of steps S113, S207 in step S115, the test result indicating that there is no abnormality in the air leakage of the sphygmomanometer 1 and that there is also no abnormality in the detection accuracy of the pressure sensor 23 is displayed on the display unit 818 as shown in FIG. 7 in step S119. The reliability with respect to the measurement result in the sphygmomanometer 1 then can be enhanced and the measurement of home blood pressure can be promoted.

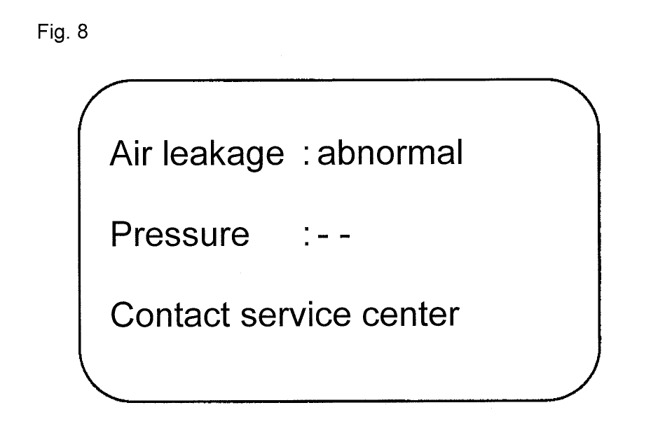

If determined that there is air leakage in the main body of the sphygmomanometer 1 in the air leakage test of steps S109, S205 in step S111, the test result indicating that there is abnormality in the air leakage of the sphygmomanometer 1 is displayed on the display unit 818 as shown in FIG. 8 in step S119. The measurement is thus not carried out using the sphygmomanometer 1 in which the air is leaking inside. The user of the sphygmomanometer 1 can quickly take measures such as requesting repair of the sphygmomanometer 1 to the manufacturing company and the like.

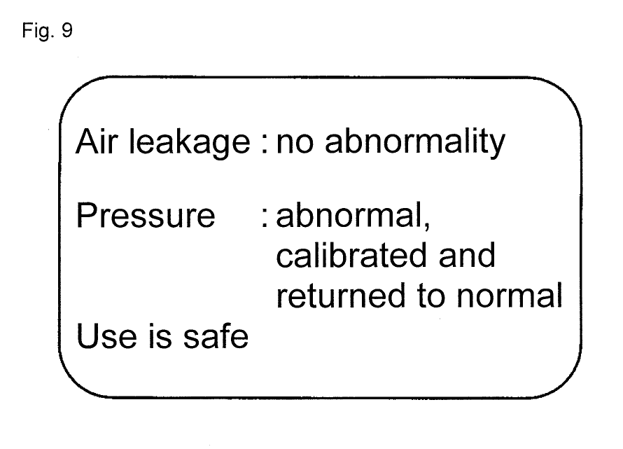

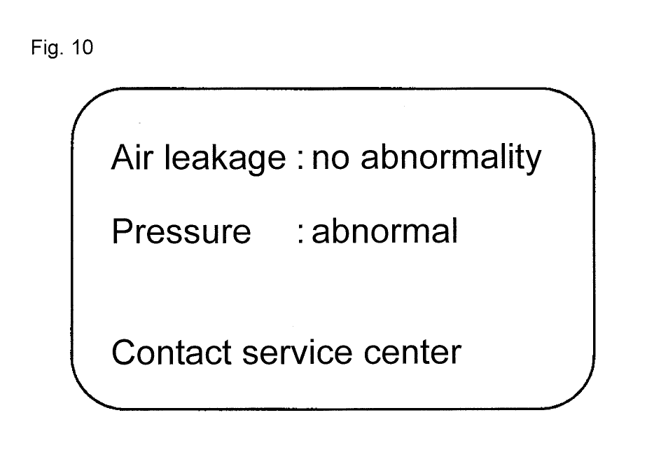

If determined that there is no air leakage in the main body of the sphygmomanometer 1 in the air leakage test of steps S109, S205 in step S111, and if the calibration operation of steps S118, S209 is carried out according to the result of the equipment difference test of steps S113, S207 in step S115, the test result indicating that although there is an abnormality in the detection accuracy of the pressure sensor 23, the pressure sensor 23 is calibrated as displayed on the display unit 818 as shown in FIG. 9 in step S119. The reliability with respect to the measurement result in the sphygmomanometer 1 then can be enhanced and the measurement of home blood pressure can be promoted. Moreover, if determined as a defect of the pressure sensor 23 when the equipment difference does not fall within a predetermined range even after the calibration operation of steps S118, S209 is carried out a predetermined number of times, the test result indicating that there is an abnormality in the measurement accuracy of the pressure of the sphygmomanometer 1 is displayed on the display unit 818 as shown in FIG. 10 in step S119. The measurement is then not carried out using the sphygmomanometer 1 having low measurement accuracy in which the sensor output of the pressure sensor 23 is inappropriate. The user of the sphygmomanometer 1 can rapidly respond such as requesting repair of the sphygmomanometer 1 to the manufacturing company.

The test of the sphygmomanometer 1 is executed when the user connects the air tube 10 and the communication cable 11 to the calibration device 8 and operates the start switch, where the pressure sensor 23 is calibrated according to the result of the equipment difference test. The calibration device 8 is a device owned by the user of the sphygmomanometer 1 along with the sphygmomanometer 1, so that consideration can be made in conducting the test at home. Consideration is also made in installing the device in stores such as pharmacies so that the user can carry the sphygmomanometer 1 to the position where the calibration device 8 is installed and conduct the test.

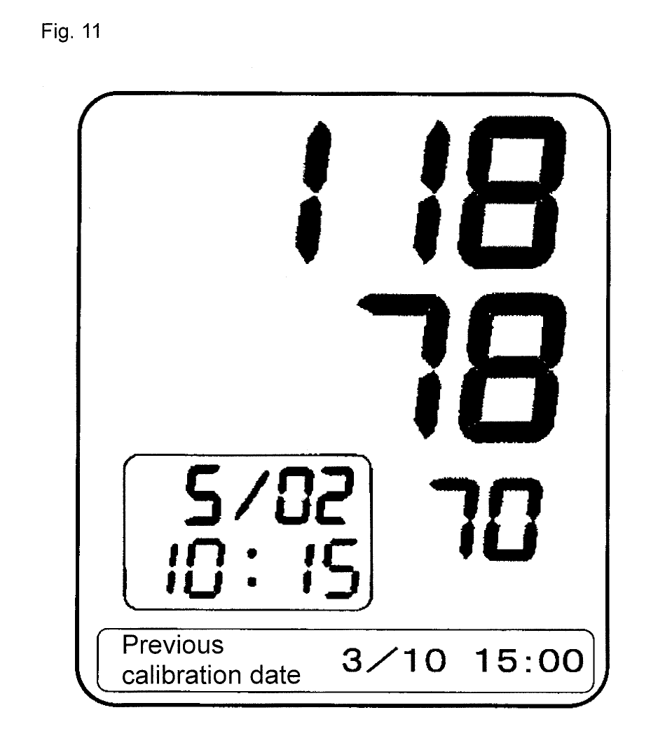

According to one or more embodiments of the present invention, the test and the calibration of the pressure sensor 23 are carried out at an interval of a constant period or an interval of a number of measurements to ensure the measurement accuracy of the sphygmomanometer 1. The CPU 40 of the sphygmomanometer 1 displays the information specifying the date and time at which the most recent calibration was performed on the display unit 4 based on the record of the calibration stored in a predetermined region of the memory 6 in step S211. As shown in FIG. 11, such display may be made when the measurement operation (not shown) is carried out and the measurement result is displayed on the display unit 4. The display may also be made after the initialization process carried out at the start of the measurement operation. Therefore, the user of the sphygmomanometer 1 can connect the calibration device 8 and perform the test when determining that a constant period has elapsed from the date and time at which the most recent calibration was performed or that the measurement is carried out at a constant number of times.

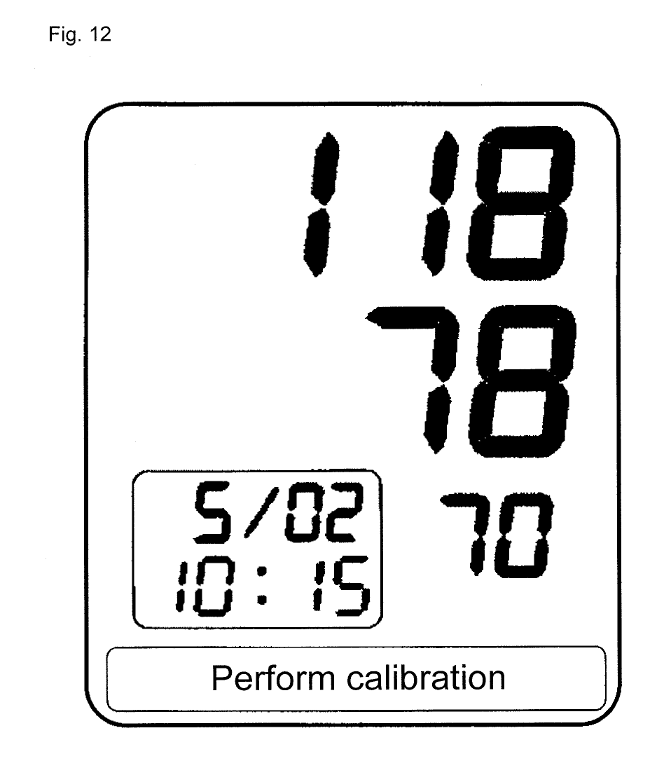

The CPU 40 of the sphygmomanometer 1 may determine as the timing to carry out the test and the calibration of the pressure sensor 23 when detecting that a predetermined period has elapsed from the most recent calibration or test based on the record of calibration or the test result stored in a predetermined region of the memory 6 in step S211, and display a screen urging the next test or calibration, as shown in FIG. 12.

In the above example, the CPU 800 of the calibration device 8 generates a control signal for storing the test result and the record of calibration in the memory 6 of the sphygmomanometer 1, and transmits the same from the communication I/F 815 to the sphygmomanometer 1 along with the information to be stored in step S119, but if the calibration system includes another device such as a server (not shown), the control signal for storing in another device may be generated and transmitted from the communication I/F 821 to another device along with the information to be stored. Another device corresponds to a server for customer management, and the like installed by the manufacturing company of the sphygmomanometer 1. In this case, the CPU 800 of the calibration device 8 transmits the information (e.g., serial number, user name registered in advance, or the like) for specifying the sphygmomanometer 1 to another device along with the above described information. In another device, the transmitted information is stored for every sphygmomanometer. Furthermore, another device may monitor the elapsed period from the date and time at which the most recent test and calibration were carried out for every sphygmomanometer, determine as the timing to perform the test and the calibration of the pressure sensor 23 when detecting that a predetermined period has elapsed, and output the same as a service guidance to the user of the sphygmomanometer 1.

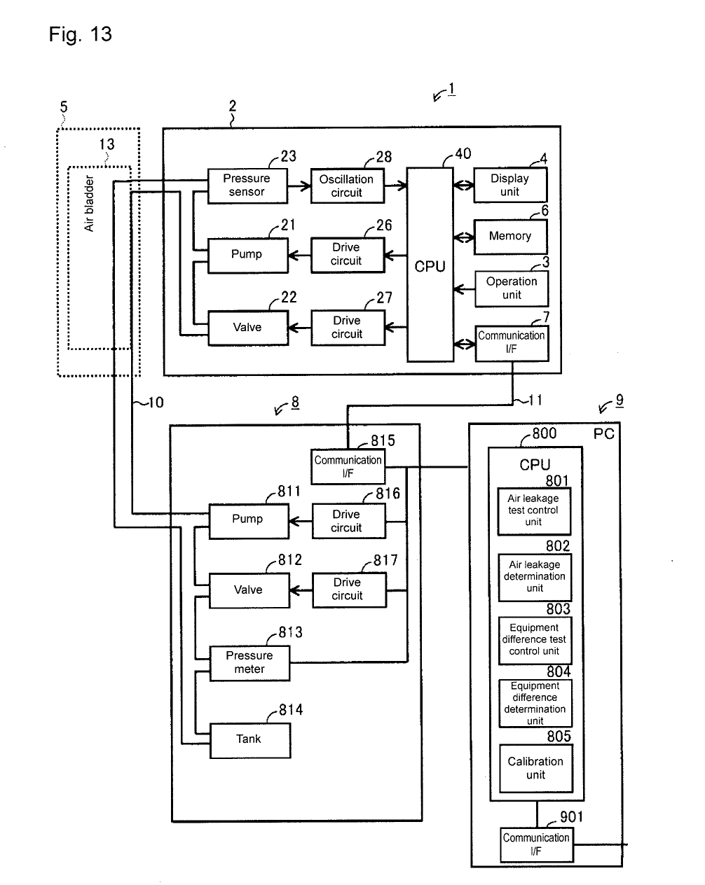

Furthermore, the calibration device 8 may include only the drive mechanism, and the control of the calibration device 8 may be carried out in another device such as a personal computer (PC) for executing the calibration program. FIG. 13 is a view showing a specific example of the configuration of the calibration system in this case, where PC 9 is arranged as another device for carrying out the control of the calibration device 8. By way of example, the configuration in which the CPU 800 is arranged in the PC 9 of each configuration of the calibration device 8 shown in FIG. 1 is shown. Each unit of the calibration device 8 is controlled by the CPU 800 of the PC 9 connected to the calibration device 8, and the test operation described above is carried out. The PC 9 further includes a communication I/F 901 for communicating with another device by connecting to the Internet, or the like.

For instance, consideration is made in the way of using such as lending the calibration device 8 to a member registered to a service for managing the health index on the Web, downloading (installing) the calibration program in the PC 9 owned by the member, and connecting the calibration device 8 to the PC 9 and then connecting the sphygmomanometer 1 to the calibration device 8 to carry out the test and the calibration of the sphygmomanometer 1 at the home of the member. The use of the calibration device 8 can be permitted only to the member by providing a usable expiration date to the calibration program. In this case, the test result and the record that the calibration was carried out obtained in the PC 9 are transmitted from the communication I/F 901 to the server or the like installed by the operator of the service and stored in the server. In the server, the timing to perform the test and the calibration of the pressure sensor 23 is determined similar to the server for customer management described above, and information indicating the same may be transmitted to the PC 9 as an e-mail.

The calibration program may be recorded in a computer readable recording medium such as a flexible disc, a CD-ROM (Compact Disk-Read Only Memory), a ROM (Read Only Memory), RAM (Random Access Memory), a memory card or the like adjunct to the computer, and provided as a program product. Alternatively, the program may be provided by being recorded in a recording medium such as a hard disc incorporated in the computer. The program may also be provided by being downloaded through the network.

The program according to one or more embodiments of the present invention may be for calling out the necessary module at a predetermined timing in a predetermined array and executing the process of the program modules provided as one part of the operating system (OS) of the computer. In this case, the relevant module is not included in the program itself and is operated cooperatively with the OS to execute the process. The program according to one or more embodiments of the present invention also includes the program that does not include such module.

The program according to one or more embodiments of the present invention may be provided by being incorporated in one part of another program such as the measurement program. In this case as well, the module included in another program is not included in the program itself and is operated cooperatively with another program to execute the process. The program according to one or more embodiments of the present invention also includes the program incorporated in another program.

The program product to be provided is installed in a program storage unit such as a hard disc, and executed. The program product includes the program itself and the storage medium in which the program is recorded.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1 sphygmomanometer
2 main body portion
3, 820 operation unit
4, 818 display unit
5 cuff
6, 819 memory
7, 815, 821, 901 communication I/F
8 calibration device
10 air tube
11 communication cable
13 air bladder
21, 811 pump
22, 812 valve
23 pressure sensor
26, 27, 816, 817 drive circuit
28 oscillation circuit
40, 800 CPU
801 air leakage test control unit
802 air leakage determination unit
803 equipment difference test control unit
804 equipment difference determination unit
805 calibration unit
813 pressure meter
814 tank

1. A management device that performs management of an electronic sphygmomanometer that detects an inner pressure change of an air bladder with a sensor and calculates a blood pressure value based on an output value of the sensor, the management device comprising:
a connecting unit that connects to the electronic sphygmomanometer;
a test unit that tests an equipment performance of the electronic sphygmomanometer while being connected to the electronic sphygmomanometer with the connecting unit;
a calibration unit that calibrates the equipment performance of the electronic sphygmomanometer according to a test result of the test unit; and
a first output unit that outputs the test result of the test unit or presence or absence of calibration in the calibration unit.

2. The management device according to claim 1,
wherein the test unit comprises:
a first test unit that performs an operation that tests for air leakage inside the electronic sphygmomanometer; and
a second test unit that performs an operation that tests for accuracy of the output value of the sensor while being connected to the electronic sphygmomanometer with the connecting unit, and
wherein the calibration unit calibrates the output value of the sensor in the electronic sphygmomanometer according to the test result of the second test unit.

3. The management device according to claim 2,
wherein the connecting unit comprises a communication unit that communicates with the electronic sphygmomanometer and a tube connecting unit that connects an air tube to configure a closed space with an interior of the electronic sphygmomanometer,
wherein the first test unit comprises an air leakage determination unit that applies a predetermined pressure with respect to the closed space while being connected to the interior of the electronic sphygmomanometer with the tube connecting unit, and determines presence or absence of air leakage inside the electronic sphygmomanometer based on a temporal change of the predetermined pressure, and
wherein the first output unit notifies on a screen when determined that air leakage is present inside the electronic sphygmomanometer by the air leakage determination unit.

4. The management device according to claim 2,
wherein the connecting unit comprises a communication unit that communicates with the electronic sphygmomanometer and a tube connecting unit that connects an air tube to configure a closed space with an interior of the electronic sphygmomanometer,
wherein the second test unit comprises:
a measurement control unit that applies a predetermined pressure with respect to the closed space while being connected to the interior of the electronic sphygmomanometer with the tube connecting unit, and outputs a control signal that measures an inner pressure of the closed space with the sensor with respect to the electronic sphygmomanometer;
an acquiring unit that acquires the inner pressure measured with the sensor from the electronic sphygmomanometer; and
a pressure determination unit that determines whether or not a difference between the predetermined pressure and the inner pressure is within a tolerable range, and
wherein the calibration unit comprises a calibration control unit that outputs a control signal that changes a relationship between the sensor signal from the sensor and the output value of the sensor with respect to the electronic sphygmomanometer when determined that the difference is outside the tolerable range by the pressure determination unit.

5. The management device according to claim 4, wherein when determined that the difference is outside the range even after the calibration is carried out a predetermined number of times by the calibration unit, the first output unit displays on a screen that the sensor has a defect.

6. The management device according to claim 1,
wherein the electronic sphygmomanometer comprises a mode that carries out a measurement and a mode that carries out a test for operation modes, and
wherein a second output unit that outputs a control signal that shifts the operation mode to the mode that carries out the test with respect to the electronic sphygmomanometer when detected as being connected to the electronic sphygmomanometer with the connecting unit is further arranged.

7. The management device according to claim 1, further comprising a first storage unit that stores information related to when the calibration is carried out by the calibration unit.

8. The management device according to claim 1, further comprising:
a communication unit that communicates with another device,
wherein the first output unit transmits the test result of the test unit or presence or absence of calibration in the calibration unit to the other device with the communication unit along with information that specifies the electronic sphygmomanometer.

9. The management device according to claim 1, further comprising a second storage unit that stores the test result of the test unit along with information that specifies the electronic sphygmomanometer.

10. A management system comprising:
an electronic sphygmomanometer that detects an inner pressure change of an air bladder with a sensor and calculates a blood pressure value based on an output value of the sensor; and
a management device, connected to the electronic sphygmomanometer, that manages the electronic sphygmomanometer,
wherein the management device comprises:
a test unit that carries out an operation that tests an equipment performance of the electronic sphygmomanometer while being connected to the electronic sphygmomanometer;
a calibration unit that calibrates the equipment performance of the electronic sphygmomanometer according to a test result of the test unit; and
an output unit that outputs the test result of the test unit or presence or absence of calibration in the calibration unit, and
wherein the electronic sphygmomanometer comprises:
a drive unit that operates the electronic sphygmomanometer according to a control signal output in the test unit;
a measurement unit that transmits a signal corresponding to the output value of the sensor to the management device with the operation; and
a changing unit that changes a relationship between the sensor signal from the sensor and the output value of the sensor according to a control signal output in the calibration unit.

11. The management system according to claim 10,
wherein the electronic sphygmomanometer further comprises:
a first storage unit that stores the blood pressure value along with information that specifies time of measurement;
a second storage unit that stores information indicating that the calibration has been carried out along with information that specifies time of calibration according to a control signal output in the output unit; and
a processing unit that performs a process of adding information indicating a measurement result of before the calibration is carried out with respect to the blood pressure value from when a calibration immediately before the calibration is carried out until the time of calibration of the information stored in the first storage unit according to the control signal output in the output unit.

12. A management method of an electronic sphygmomanometer in a management system comprising:
an electronic sphygmomanometer that detects an inner pressure change of an air bladder with a sensor and calculates a blood pressure value based on an output value of the sensor; and
a management device, connected to the electronic sphygmomanometer, that manages the electronic sphygmomanometer,
wherein the method comprises the steps of:
having the management device detect connecting with the electronic sphygmomanometer, and carry out an operation that tests an equipment performance of the electronic sphygmomanometer while being connected to the electronic sphygmomanometer;
the electronic sphygmomanometer operating according to a control signal output from the management device in a step of carrying out the operation that tests the equipment performance;
the electronic sphygmomanometer transmitting a signal corresponding to the output value of the sensor to the management device with the operation;
the management device determining the equipment performance of the electronic sphygmomanometer based on at least one of the signal transmitted from the electronic sphygmomanometer and the output value detected in the connected state;
the management device calibrating the equipment performance of the electronic sphygmomanometer according to the determination;
the electronic sphygmomanometer changing a relationship between the sensor signal from the sensor and the output value of the sensor according to a control signal output from the management device in the step of calibrating the equipment performance; and
the management device outputting the test result or presence or absence of calibration of the output value of the sensor.
